(19) United States
(12) Patent Application Publication
(10) Pub. No.: US 2010/0190236 A1
(43) Pub. Date: Jul. 29, 2010

(54) System for Producing Microorganisms

(76) Inventor: Dominique Delobel, Carvin (FR)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/666,420

(22) PCT Filed: Jul. 2, 2008
(86) PCT No.: PCT/FR2008/051228

§ 371 Date: Jan. 20, 2010

(30) Foreign Application Priority Data
Jul. 3, 2007 (FR) 0704847

(51) Int. Cl. C12N 1/00; C12M 1/00

(52) U.S. Cl. 435/243; 435/292.1

(57) ABSTRACT

The invention relates to a device for producing biomass containing any species of microalgae and cyanobacteria, and more generally any microorganisms whose growth depends on photosynthesis in a liquid medium.

Moreover, the device includes two confinement housings imbedded and built in situ:

i) Said first confinement housing includes a photobioreactor including two juxtaposed inter-dependent sub-devices consisting of a) a culture chamber (1) and b) a technical block (2) juxtaposed to said culture chamber (1) at one end (4) thereof at least.

ii) Said second confinement housing (5) contains the technical blocks (2) of several juxtaposed photobioreactors.

The invention also relates to a method for producing said biomass using said device.

The present invention relates to a device for producing biomass of any species of microalgae and cyanobacteria, and more generally any microorganisms whose growth depends on photosynthesis in a liquid medium.

The invention also relates to a method using said device.

Said device and said method are aimed in particular at industrialists from the food processing, chemical, pharmaceutical, cosmetics and energy production industries.

The devices for producing such microorganisms, apart from harvesting in a natural medium, known to those skilled in the art belong to two main categories:

First, closed systems or photobioreactors, either at laboratory or industrial scale, have a small capacity, generally less than one thousand liters. They operate with the aid of artificial sources of light, heat, and mechanical agitation. They are used for the intensive or super-intensive production of small amounts of biomass of very high quality in terms of purity or composition. Said quality is attained through the continuous control of virtually all the culture parameters and by rigorously restricting contamination of the culture by pollutants such as macro- or micro-organisms, dust and gases.

Such devices give priority to the quality of the biomass produced without being able to produce the large quantities required for industrial applications. Their high manufacturing and operating costs, particularly energy and maintenance costs, make their widespread implantation unrealistic from an economic standpoint.

Secondly, open systems are composed of open ponds having a medium or large capacity, from several thousand to several million liters. They operate primarily with the aid of natural resources such as the sun, which supplies light and heat, hence a limited control of the culture parameters which depend on these factors. The ponds are sometimes covered, usually in a greenhouse so as to minimize contamination, and to improve temperature control. Covering ponds of more than one thousand square meters with a capacity of approximately two hundred cubic meters requires the use of imposing structures which suffer from the drawback of significant wind exposure, or else the use of smaller structures which require the installation of support structures inside the ponds themselves. These solutions for covering open ponds remain problematic and costly, be it in terms of their installation or their maintenance.

Such devices make up for their low or moderate productivity by a large size. They give priority to the quantity of biomass produced. Their drawback is that they only imperfectly satisfy the quality requirements of the food processing or pharmaceutical industries users of biomass.

So as to alleviate these different problems, the invention discloses a device for producing biomass containing any species of microalgae and cyanobacteria, and more generally any microorganisms whose growth depends on photosynthesis in a liquid medium, allowing the production of large quantities of biomass such as can be obtained in open systems, to which it is near in size, with the degree of quality specific of closed photobioreactor systems.

The general or particular embodiments of the invention further permit a cost savings of the technical means and materials to be employed for setting up a production site.

According to a major aspect of the invention, said device has two imbedded confinement housings built in situ:

i) The first confinement housing includes a photobioreactor composed of two juxtaposed inter-dependent sub-devices consisting of:

a) an airtight and watertight culture chamber, the upper part thereof being permeable to visible light, and with an internal volume ideally greater than fifty cubic meters, built longitudinally on flat profiled ground;

b) a technical block juxtaposed to said culture chamber at at least one of its ends, said technical block including all the technical equipment for the conduct and control of the biomass cultures, thermal regulation and circulation of all the fluids used.

ii) The second confinement housing includes the technical blocks of several juxtaposed photobioreactors, said second housing containing all the technical means necessary for normal photobioreactor operation, on the one hand, and for harvesting and processing the recovered biomass, on the other hand.

Preferably, said biomass is harvested in such a way as to maintain the production in a contained and controlled environment until the final stage of packaging, prior to its subsequent use such as defined by the user of the device.

According to another aspect of the invention, the device has a U-shaped culture chamber equipped with two juxtaposed free ends, said ends are connected to the technical block of the photobioreactor.

According to another aspect of the invention, the technical block of the photobioreactor is positioned below ground level and below the level of the culture chamber.

According to another aspect of the invention, the culture chamber contains air and gases which are maintained in positive pressure.

According to another aspect of the invention, and according to a particular embodiment, the upper cover of said culture chamber (1) is made of a flexible material, and the level of positive pressure of air and gases is adjusted so as to hold said upper cover aloft over its entire surface and keep it away from the free surface of the liquid medium.

According to another aspect of the invention, said culture chamber has a channel height and width, said height being less than 25% of the channel width.

According to another aspect of the invention, and according to a particular embodiment which involves the use of a flexible material for covering the culture chamber, the device can be secured in case of high winds by momentarily stopping the positive pressure of air and gases contained in the culture chamber. The cover settles onto the surface of the liquid medium contained in the culture chamber, considerably reducing the wind exposure of the device which is thus secured.

According to another aspect of the invention, and according to a particular embodiment which involves the use of a flexible material for covering the culture chamber, the securing of the device in the event of a hurricane can be reinforced by placing a layer of water on top of the upper cover of the culture chamber, previously settled as described earlier. Resumption of the positive pressure of air and gases contained in the culture chamber causes said layer of water to flow back and be pushed towards the edge of said cover.

According to another aspect of the invention, said culture chamber may be positioned on a natural or artificial expanse of shallow water.

It is another object of the invention to provide a method for producing biomass using said device.

According to another major aspect of the invention, the method comprises at least one of the following phases:
Circulation of the culture medium between the two channels of the culture chamber,
Continuous positive pressure of the culture chamber,
Supply of solid, liquid or gas inputs,
Continuous or intermittent control of the cultures by means of any measuring devices,
Removal of mineral and organic waste out of the culture chamber,
Transient removal and reintroduction of the culture medium out of and into the culture chamber for the purposes of harvesting the produced biomass,
Thermal regulation of the liquid content in the culture chamber,
Containment of the device constituting the work area,
Processing and packaging the harvested biomass.

The invention remedies the aforementioned drawbacks in that it allows to produce large quantities of biomass such as obtained in open systems to which it is near in size, all while improving productivity, and to attain the high quality peculiar to closed systems.

The general or particular embodiments of the invention further permit a savings in terms of the technical means to be used for setting up a production site.

The aforementioned aspects of the invention presented in a non-limiting manner will become more apparent from the following description of a preferred embodiment, and of particular embodiments. Said description refers to the accompanying drawings:

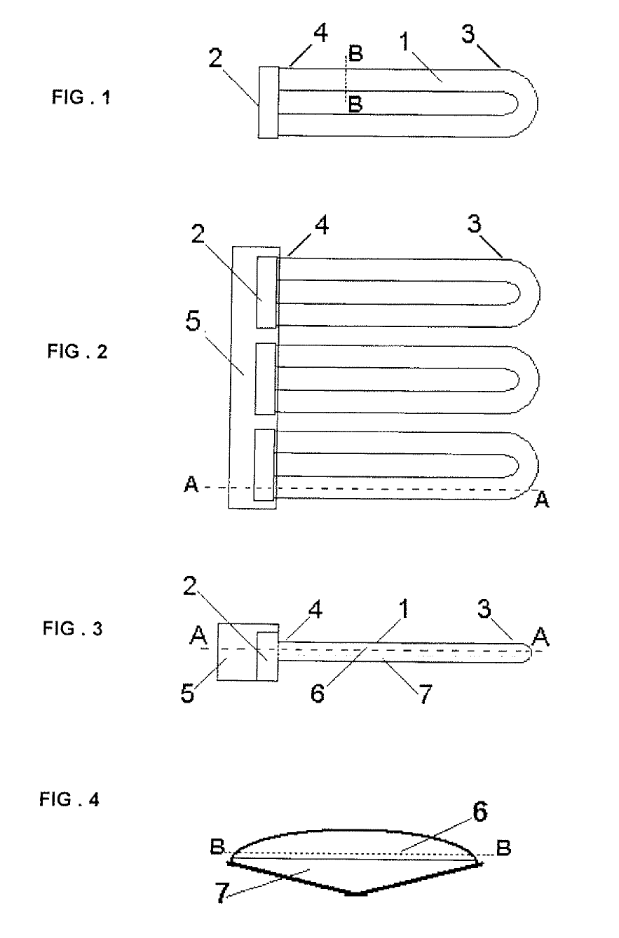

FIG. 1 is a top view of the arrangement of photobioreactor components: culture chamber and technical block.

FIG. 2 is a top view of the layout of a production unit including several photobioreactors.

FIG. 3 is a cross-sectional view of A-A in FIG. 2.

FIG. 4 is a cross-sectional view of B-B in FIG. 1 referring to one of the particular embodiments of the invention.

FIGS. 1 and 2 depict a preferred embodiment of the invention.

The invention is based on the construction in situ, on bare, levelled, flat and horizontal ground, obtained by appropriate civil engineering works known to those skilled in the art, of two imbedded confinement housings.

Said first confinement housing is composed of an independent unit forming a photobioreactor of large size which consists of two juxtaposed inter-dependent sub-devices:
a culture chamber (1) having a volume preferably greater than fifty cubic meters built longitudinally on flat profiled ground.
a technical block (2), juxtaposed to said culture chamber (1) at one end (4) thereof, to which it is connected in an impermeable manner. Said technical block (2) includes all the technical equipment for the conduct and control of the biomass cultures, thermal regulation and circulation of all the fluids. It constitutes the only point of entry and exit of all fluids and materials to and from said photobioreactor.

Said first confinement housing contains only the growing biomass and the components of the culture medium thereof. Said chamber is not accessible to site personnel for current operations.

Said second confinement housing (5) includes the technical blocks (2) of several juxtaposed photobioreactors. Said second housing (5) contains all the technical means necessary for normal operation of said photobioreactors, on the one hand, and for harvesting and processing the recovered biomass so as to maintain the production in a contained and controlled environment until the final stage of packaging, prior to its subsequent use such as defined by the user, on the other hand. Said second confinement housing (5) is normally accessible to site personnel for current operations.

According to the general embodiment of the invention, each photobioreactor is built as follows:

Said U-shaped culture chamber (1) is built on bare, flat, horizontal, levelled, profiled ground by civil engineering works in accordance with the rules of the art.

The construction of the support slab involves the tracing and levelling of two parallel channels of equal length, spaced and wide, for example according to different embodiments of the invention from less than one to a few dozen meters. Said two channels are connected by an elbow at one of their ends (3).

The levelling of each channel is carried out so as to obtain a longitudinal slope of zero to ten percent and a widely flared V-shaped cross-sectional profile having a double axial slope of zero to twenty degrees as illustrated in FIG. 4.

According to particular embodiments of the invention, the longitudinal slopes can be either in the same direction, or in opposite directions.

Said culture chamber (1) of each photobioreactor has a total overall length comprised between twenty and two thousand times its width. Its height, which varies according to the particular embodiments, is less than twenty-five percent of its width such as for example one meter for a channel width of four meters.

Said culture chamber (1) intended to contain said liquid culture medium is built by any appropriate means in accordance with the following rules:

Said culture chamber (1) takes the form of a covered channel or tube, U-shaped, airtight and watertight over its entire length, permeable to visible light in its upper part, and able to withstand an internal positive pressure of air or gas of less than five hundred millibars. The upper part of said chamber (1) can be made of any rigid or flexible material, consisting of either a single piece or by juxtaposing a plurality of pieces. Said upper part is built so as to allow the free circulation of fluids in liquid phase and/or gases through the elbow (3) of the culture chamber (1) between the two straight parts forming the U branches or channels of said device.

The liquid content (7) of said culture chamber (1) occupies from ten to seventy percent of the lower part of the cross section of said culture chamber (1), such as illustrated in FIG. 4. The free circulation of air and gases (6) is freely ensured over the entire length of said culture chamber (1), in the upper part (headspace) of said cross section not occupied by said liquid content (7).

Irrespective of the embodiment of said culture chamber (1), it is maintained under internal positive air pressure, by any appropriate technical means such as compressor, turbine, fan. Said positive pressure avoids, in the event of a leak in one of the walls of said culture chamber (1), the entry of external fluids, gases or liquids, through said leaks into said culture chamber (1).

According to particular embodiments of the invention, said culture chamber (1) can be built by any assembly of flexible or rigid materials, or by assembly of flexible sections associated with rigid sections.

According to a particular embodiment of the invention, said culture chamber (1) can be positioned entirely on a natural or artificial expanse of shallow water.

In the case where a flexible material is used to construct the upper part of said culture chamber (1), said upper part is held away from the free surface of the liquid content (7) by adjusting the positive pressure of the internal air. Said positive pressure holds said upper part aloft over the entire surface of said culture chamber (1).

In the case where a flexible material is used to construct the upper part of said culture chamber (1), said upper part is sized and positioned so that when deflated it will rest without tension on the bottom of said culture chamber (1) empty of all liquid content (7).

Thus said upper part can be intentionally flooded, when necessary, and covered with a layer of water several centimeters deep which secures said device against the destructive effects of high winds.

Each of said two juxtaposed open ends (4) of said culture chamber is connected, permanently and impermeably, to said technical block (2).

Said technical block (2) according to the inventive embodiment is a compact device the dimensions thereof, which vary according to the particular embodiments, are comprised within the following limits: the length thereof is equal to no more than the width of the two support channels of said culture chamber (1) increased by that of the space which separates them, the width thereof is less than three meters and the internal volume thereof is comprised between one and twenty per thousand of the maximum volume of said culture chamber (1) to which it is connected.

As illustrated in FIG. 3, said technical block (2) is built in such a way that, according to the particular embodiments of the invention, the base thereof is located between zero and three meters below the level of the supporting ground of said culture chamber (1) and the top thereof is located at a height greater than that of said culture chamber (1).

Said technical block (2) forms a closed, integral whole, which contains all the technical means necessary for proper operation of the photobioreactor. It carries out the following functions:
circulation of the culture medium between the two channels of said culture chamber (1) with the aid of any suitable devices including non-limiting examples such as channel, tube or hose, valves, pumps, “airlift”, tanks or any other device adapted to a particular embodiment,
continuous positive pressure of said culture chamber (1) and control thereof by any suitable device including non-limiting examples such as fan, blower, compressor, vent, manometer,
input of the solid, liquid or gaseous components required for carrying out the cultures via suitable conduits, tubes and valves known to those skilled in the art,
continuous or intermittent control of the cultures by means of any measuring devices including non-limiting examples such as thermometer, flowmeter, pH meter, conductimeter, sensors,
removal of mineral and organic wastes outside of said culture chamber (1) by means of any suitable device including non-limiting examples such as pump, filter, settler, centrifuge,
transient removal of said culture medium outside of said culture chamber (1) for purposes of harvesting the produced biomass, and reintroduction thereof into said culture chamber (1) by means of any device adapted to each particular embodiment, such as pump, siphon, bypass channel,
thermal regulation of said liquid contained in said culture chamber (1) by any system for producing cold or heat and immersed heat exchanger adapted to the particular embodiment.

According to the general embodiment of the invention illustrated in FIG. 2, the second confinement housing (5) consists of a structure, the size and shape thereof adapted to the particular embodiments, constructed according to the rules of the art. Said second housing (5) occupies an area less than five percent of that of the set of photobioreactors it contains.

In addition to said technical blocks (7), said second housing contains the devices necessary for carrying out the following functions:
containment of the work area by installation of devices known to those skilled in the art and commonly used in the pharmaceutical industry including non-limiting examples such as: fixed frame window, entry-exit port, positive pressure packaging and air filtration system,
harvesting of said biomass content by any suitable device known to those skilled in the art, including non-limiting examples such as: rotary drum filter, vibrating filter screen, vacuum filter, mobile cloth filter, centrifuge,
supplying said photobioreactors with input of liquids, gases or solids, which presumes that the required product storage units, gas and liquid conduits are adapted to the operating requirements according to the particular embodiments,
processing said harvested biomass and packaging it in fresh, frozen or dehydrated form by means of technical devices including non-limiting examples such as: press, dryer, evaporator, dessicator, centrifuge, freeze-drying system, tunnel freezer, bagging machine, cold room, spray tower, mill, and the like.

In order to conform to the spatial positioning, previously described, of said technical block (2) relative to said connected culture chamber (1), it is necessary according to the particular embodiments of the invention to either build said second confinement housing (5) below ground level and where said culture chambers (1) are positioned, or to arrange in said housing, at the location of each technical block (2), a pit whereby it can be positioned.

According to a preferred particular embodiment of the invention, said culture chambers (1) are constructed from a sheeting of flexible watertight and airtight material, permeable to visible light and inflatable while being able to withstand an internal positive pressure of air or gas of less than five hundred millibars.

A translucid agricultural polyethylene sheeting with a minimal thickness of twenty hundredths of a millimeter, obtained by the known industrial process of extrusion, is a non-limiting example of a material adequate for the construction of said device.

The length of said sheeting is comprised between twenty and two thousand times its width. The length required for this particular embodiment of the invention can be obtained by placing end-to-end and assembling by welding or gluing by any suitable technical means known to those skilled in the art several sheets of identical width and having lengths less than that required.

According to the general embodiment of the invention, a lining of the levelled support slab such as described earlier may be constructed according to the rules of the art by using any appropriate material, including non-limiting examples such as: sand, concrete, asphalt, wood, tiles. Said lining provides the principal function of protecting the physical integrity of said flexible sheeting, known to be fragile.

Said protection may be reinforced by inserting a protective layer between said sheeting and said support slab, lined or not. Said layer may be composed of plastic film, felt, a geomembrane, a geotextile or any other substance or material adapted to said use.

The mass of liquid which forms the culture medium after being introduced in the sheeting ensures, given the cross-sectional profile of the support slab, that the device is anchored to the ground which is essential.

The culture chamber is maintained under slight positive pressure of air or gas of approximately a few dozen millibars produced by a suitable air compression device located in said technical block (2) juxtaposed to said culture chamber (1). The internal positive pressure is continuously adjusted so as to allow said culture chamber (1) to deform under the effect of wind or a pressure exerted by any means on the external surface of said sheeting.

The inventive device is intended in particular for equipping production sites for large biomasses of photosynthetic aquatic microorganisms, with an operational area equal to one hectare at least.

It should be noted that the aforementioned device may accommodate any component not described and keeping in line with the invention.

The inventive embodiments exemplified above were chosen for their practical nature. It would not however be possible to provide an exhaustive list of all the embodiments encompassed by the invention.

In particular, any of the means described may be replaced by an equivalent means without departing from the scope of the present invention.

1-11. (canceled)

12. A device for producing biomass comprising two confinement housings imbedded and built in situ:
i) the first confinement housing comprising a photobioreactor composed of two juxtaposed inter-dependent sub-devices consisting of:
a) an airtight and watertight culture chamber (1), permeable to visible light in its upper part and built longitudinally on flat profiled ground; and
b) a technical block (2) juxtaposed to said culture chamber (1) at at least one of its ends (4), said technical block (2) containing all the technical equipment for the conduct and control of the biomass culture, thermal regulation and circulation of all the fluids used; and
ii) the second confinement housing (5) includes said technical blocks (2) of several juxtaposed photobioreactors, said second housing (5) containing all the technical means required for normal operation of said photobioreactors and for harvesting and processing said biomass so as to maintain it in a contained environment until the final stage of packaging.

13. The device according to claim 12, wherein the culture chamber (1) is U-shaped at the two juxtaposed free ends (4), said ends (4) are connected to the technical block (2) of said photobioreactor.

14. The device according to claim 12, wherein said technical block (2) of said photobioreactor is positioned below ground level and below the level of said culture chamber (1).

15. The device according to claim 12, wherein air and gases contained in said culture chamber (1) are maintained under positive pressure.

16. The device according to claim 15, wherein the upper cover of said culture chamber (1) is made of a flexible material and the level of positive pressure of air and gases (6) contained in said culture chamber is adjusted so as to hold the entire surface of the upper cover aloft and keep it away from the mass of liquid content (7) of said culture chamber (1).

17. The device according to claim 12, wherein said culture chamber (1) has a channel height and width, said height being less than 25% of said width.

18. The device according to claim 16, wherein the flexible upper cover of the culture chamber (1) may be momentarily settled on the liquid content (7) by stopping the positive pressure of the headspace air and gases (6), said device now being secured by the reduction of its wind exposure.

19. The device according to claim 18, wherein the flexible upper cover of the culture chamber (1) is momentarily covered with a layer of water, the device now being secured.

20. The device according to claim 12, wherein said culture chamber (1) is positioned on a natural or artificial expanse of shallow water.

21. The device according to claim 12, wherein said airtight and watertight culture chamber (1) has an internal volume of greater than about fifty cubic meters.

22. A method for producing biomass comprising culturing photosynthetic biomass producing microorganisms in a device according to claim 12.

23. The method for producing biomass according to claim 22, wherein said method comprises at least one of the following phases:
circulation of the culture medium between the two channels of said culture chamber (1);
continuous positive pressure of said culture chamber (1);
supply of solid, liquid or gas inputs;
continuous or intermittent control of the cultures by any measuring devices;
removal of mineral and organic waste outside of said culture chamber (1);
temporary removal and reinjection of said culture medium out of and into said culture chamber (1) for the purposes of harvesting said produced biomass;
thermal regulation of said liquid content (7) in said culture chamber (1);
containment of said device constituting the work area; or
processing and packaging of said harvested biomass.
